(19) United States
(12) Patent Application Publication
(10) Pub. No.: US 2008/0221803 A1
(43) Pub. Date: Sep. 11, 2008

(54) Methods of predicting treatment outcome in chronic periodontitis patients

(76) Inventor: Martin Levine, Norman, OK (US)

Correspondence Address: DUNLAP CODDING, P.C., PO BOX 16370, OKLAHOMA CITY, OK, 73113, US

(21) Appl. No.: 12/012,048

(22) Filed: Jan. 31, 2008

Related U.S. Application Data
(60) Provisional application No. 60/898,569, filed on Jan. 31, 2007.

(51) Int. Cl. G01N 33/48

(52) U.S. Cl. 702/19

(57) ABSTRACT

The invention is a new method for predicting when subjects having chronic periodontitis will fail or succeed conventional standard therapy. The method uses measures of the plaque cadaverine fraction with additional measurements including age, fraction of gingival margins that bleed on probing (BOP), and clinical attachment level (CAL in mm) to calculate discriminant function equations for calculating individual score sets. When CF is above 0.45 in periodontitis patients, it is predicted that most available therapies with which the periodontitis patient can be treated will fail, including a conventional standard treatment or an aggressive combined antibiotic/oral hygiene therapy.

### CROSS-REFERENCE TO RELATED APPLICATIONS

The present application claims the benefit under 35 U.S.C. 119(e) of U.S. Provisional Application Ser. No. 60/898,569, filed Jan. 31, 2007, the entirety of which is hereby expressly incorporated herein by reference.

### STATEMENT REGARDING FEDERALLY SPONSORED RESEARCH OR DEVELOPMENT

Some aspects of this invention were made in the course of Grant 5 R21 DE 14583 awarded by the National Institutes of Health and Dental & Craniofacial Research and therefore the Government has certain rights in some aspects of this invention.

### BACKGROUND

Chronic periodontitis is a loss of tooth support that occurs in about 25% of US adults aged 30 years and up. It is indicated by an increase in clinical attachment level (CAL). To measure CAL, it is first necessary to measure the pocket probing depth, the distance in mm from the free gingival margin to the base of a sulcus or pocket. If the cemento-enamel junction is apical to the free gingival margin, CAL is obtained by subtracting the distance in mm between the cemento-enamel junction and free gingival margin from the pocket depth otherwise it is added to the pocket probing depth. The mean pocket probing depth (PD) is also important because pockets greater than 2 mm may act as a focus of infection that interferes with therapy.

Chronic periodontitis is associated with gingival inflammation (gingivitis) and a teeth-adherent bacterial biofilm (plaque). Most young adults exhibit mild gingivitis (Goodson et al. 2004) but no periodontitis and little plaque. If young adults abolish all oral hygiene practices, plaque becomes visible at the gingival region of teeth within four days (Furuichi et al. 1992). The amount of gram negative bacteria and spirochetes in the plaque also increases, and by 3 weeks gingivitis is clinically obvious (Loe et al. 1965). Prior to gingivitis appearing, sub-clinical inflammation is evident as gingival crevicular fluid (Griffiths et al. 1997, Loe & Holm-Pedersen 1965), a plasma or serum protein-rich exudate from the gingival crevice (sulcus). Other crevicular fluid components, including saccharides, amino acids, vitamins and minerals, also support the bacterial growth required for plaque development.

The bacteria hydrolyze crevicular fluid proteins to amino acids which they anaerobically metabolize to ammonia, short chain fatty acids and carbon dioxide (Marsh 2003, Niederman et al. 1996). Ammonia creates an alkaline environment in the sulcus (Bickel et al. 1985) where it precipitates calcium phosphate from the crevicular fluid onto teeth surfaces (calculus) and traps the bacteria beneath. This persistent infection elevates systemic inflammatory mediators, predisposing the affected individual to cardiac or cerebrovascular events (Loos et al. 2000). Better prevention of periodontitis would clearly benefit human health. Nevertheless, it is not clear how the plaque induces periodontitis, and why it should occasionally not respond to plaque control (Colombo et al. 1998, Haffajee et al. 2004).

Studies indicate that various agents from plaque, including short chain fatty acids, inhibit the growth of mammalian cells in culture (Levine 1985). When antibodies that partially abrogated the growth inhibition were made (Levine & Miller 1991), they were found to cross-react with Eikenella corrodens (McAnally & Levine 1993) by binding to an 80 kDal protein antigen on Western immunoblots (Levine & Miller 1996). This antigen was identified as lysine decarboxylase, an enzyme which converts lysine to cadaverine and carbon dioxide. Lysine decarboxylase is not made by the host and it inhibits mammalian cell growth by depleting lysine, an essential amino acid in culture medium (Levine et al. 2001).

At the base of a gingival sulcus, the junctional epithelial attachment is composed of basal keratinocytes which are adherent to the tooth surface on one side and the gingival stroma on the other (Schroeder & Listgarten 1977). Unlike oral mucosa from which it is derived, the junctional epithelial cells differentiate very little and remain permeable to interstitial fluid. Its dentally-attached cells maintain a proliferative basal phenotype and require the nutrients from interstitial fluid to maintain that phenotype in the absence of underlying capillaries (Salonen 1994).

E. corrodens is a major source of lysine decarboxylase in plaque (Holmes et al. 1995) where it appears within a few hours after cessation of oral hygiene (Li et al. 2004). Lysine decarboxylase may therefore cause the coronal, dentally attached cells to become lysine-starved, release inflammatory mediators and induce the crevicular fluid exudate (Levine et al. 2001). The extent of dentally attached cell lysine depletion may be indicated by measuring the cadaverine mole fraction of lysine plus cadaverine in plaque (“Plaque Cadaverine Fraction”—CF). Although the crevicular fluid exudate may contain sufficient lysine to reverse DAT cell lysine depletion, the greater protein content of the exudate than saliva likely promotes plaque development (Marsh 2003, Socransky & Haffajee 2005).

Standard therapy for periodontitis comprises removing the plaque and its calcified deposits (calculus) by scaling and root planing the teeth (SRP). In addition, periodontal surgery is necessary to remove deepened pockets, or repair a locally excessive loss of periodontal attachment. Finally balancing heavy occlusal contacts may be necessary to reduce a predisposition to recurrent gingival inflammation (Parameters of care supplement 2000). Antibiotic therapy is discouraged, and only provided if the patient exhibits an increase in mean CAL within a year of completing the above therapy.

A maintenance program, twice-daily home care and professional SRP every 3 months, is essential for minimizing plaque and calculus re-development. In general, “treatment failure” expresses as continued attachment loss in spite of adequate treatment and proper oral hygiene. More specifically, standard therapy is said to have failed if a patient exhibits an increase in mean CAL twice within a year following therapy (Colombo et al. 1998). The greater the CAL pre-therapy, the greater is this likelihood (Haffajee et al. 2004, Nieminen et al. 1995). Attachment loss within 3 months of completing the professional therapy may also predict therapeutic failure (Haffajee et al. 1997).

Therapeutic fail may occur when the patient fails to carry out proper oral hygiene. Or therapeutic failure may occur in spite of home care properly conducted by the patient. Only the latter is a response outside the patient's control. For example, cigarette smoking associates with more attachment loss, but not necessarily with a therapeutic failure (Colombo et al. 1998, Haffajee et al. 1997, Levine et al. 2002). Many factors appear related to failure to periodontal therapy, including extent of disease prior to therapy, type of therapy provided (nonsurgical vs. surgical, with or without antibiotics, etc.), tooth type and furcation involvement, species and strains of microflora, degree of host response (particularly immune response), and whether the patient smokes. Therapeutic failure has also been referred to in the field as a “refractory response”.

The ability to identify subjects who are likely to fail to respond to periodontal therapy (i.e., who have a high probability of therapeutic failure) would be valuable in determining a patient's potential for treatment failure at the outset, or within a few months of beginning therapy. Similarly, the ability to identify patients who would successfully respond to treatment would also be valuable.

NHANES III (Third National Health and Nutrition Examination Survey, 1988-94) is the most accurate sampling of periodontal disease in the US (Albandar et al. 1999). It represents 103 million dentate, non-institutionalized Americans aged 30 years and older from the 1990 US Census. Disease prevalence is the fraction of the population who had a probing pocket depth >4 mm. Disease extent (severity) is the mean percentage of teeth so affected. Minimal loss of tooth support (mean CAL<2 mm) occurs in 60% of affected adults, slight to moderate loss (mean CAL 2-4 mm) in 30%, and moderate to severe loss (mean CAL>4 mm) in 10% (Albandar et al. 1999).

It can be calculated (Colombo et al. 1998) that about 15% of adults with slight to moderate attachment loss and about half of those with moderate to severe loss are likely to fail therapy. This translates to about approximately 5 million US adults with mild to moderate periodontitis and another 5 million of the adults with severe periodontitis. However, patients actively seeking therapy will tend to be in the more severe half of those with mild to moderate disease, or already have severe disease, a total of 40 million adults of whom 10 million (25%) will respond poorly to therapy.

Previous investigators utilized multiple tooth loss as the outcome of a poor response to therapy (Goldman et al. 1986, Hirschfeld & Wasserman 1978, McFall, Jr. 1982). It was found that 20-25% of patients who received therapy and regular maintenance visits for at least 15 years lost 5 or more teeth. This fraction of unsuccessfully treated patients corresponds to the fraction derived from NHANES III after adjusting for the Colombo study findings (Colombo et al. 1998).

Gingivitis is detected by looking for bleeding on gently probing the sulcus (BOP). The greater the fraction of gingival surfaces with BOP (measured as described in section 39), the greater the chance of future attachment loss. If BOP exceeds 65%, meta-analysis indicates that CAL will have increased within a year with a specificity of 76% and a sensitivity of 44% (Armitage 1996). Detecting pathogenic bacteria (Boyer et al. 1996, Loesche et al. 1990) or potentially destructive host responses (Offenbacher et al. 1993, Persson et al. 1995) has not been commercially successful. The sensitivity and specificity of these tests are not sufficiently greater than those of BOP to justify the cost. Moreover, neither BOP nor the above tests distinguish between unsuccessfully treated patients who have a biological problem from those who fail to follow home care procedures.

More sophisticated studies that do distinguish patients who respond poorly to therapy (treatment failure) and have a biological problem require assaying 85 bacterial taxa for the number of species exhibiting serum antibody >50 μg/ml. In addition, plaque from many teeth surfaces is assayed for certain bacteria being above a certain fraction of total bacteria by DNA-DNA hybridization (Colombo et al. 1999). Obtaining these measurements requires levels of expertise and expense that are not commercially viable.

A method of predicting when a patient with periodontitis will fail (or succeed) under standard therapeutic treatment would be of great benefit to millions of persons. It is to this goal that the present invention is directed.

### SUMMARY OF THE INVENTION

The invention is a new method that uses standard clinical measurements and a whole mouth plaque sample to predict when chronic periodontitis patients who are given a standard therapeutic treatment are more likely to have a successful outcome or a failure outcome to the treatment regimen.

### BRIEF DESCRIPTION OF THE DRAWINGS

FIG. 1 shows the elution positions of lysine, cadaverine & putrescine from Spherogel. Arrow indicates the change to second buffer (section 34). The elution times and amounts of various amino acids and amines, including lysine and cadaverine, were determined by passing standard solutions through the column. In the first plaque sample (top), the lysine concentration is low and the cadaverine concentration is high. In the second sample (bottom), the lysine concentration is high and the cadaverine concentration is low. Plaque cadaverine fraction (CF)=[nmol cadaverine]/[(nmol lysine+nmol cadaverine)]. The sharp peak that eluted before cadaverine indicates putrescine, a diamine from ornithine.

FIG. 2 shows the prediction of healthy, unsuccessfully treated (treatment failure) or successfully treated patient groups by discriminant analysis using CF, CAL, Age and BOP. Symbols: ∘—healthy, □—successfully treated, ▴—unsuccessfully treated (treatment failure). Centroids are indicated by a large asterisk. Algorithms that define each individual point on the 2-dimensional canonical structure matrix and their statistical significance are given in the text. Gray triangles—2 periodontitis subjects misclassified as successfully treated; gray square—1 periodontitis subject misclassified as unsuccessfully treated.

FIG. 3 is a graph of cadaverine fraction against bleeding on probing in healthy subjects pre-therapy. CF is graphed against pre-therapy BOP. Healthy subjects had minimal gingivitis (BOP≦11%) and a logarithmic relationship to cadaverine fraction. (CF=0.26*Log(BOP)+0.29; adjR2=0.67, p<0.03). The change in the curve to a dotted line is a back-extrapolation to show that BOP approaches zero when CF is zero. Symbols: ∘—healthy, —healthy but not followed for a year.

FIG. 4 shows that cadaverine fraction decreases as attachment level increases in periodontitis subjects. CAL was measured pre-therapy and CF was measured post-therapy. Regression equation: CAL=5.83−4.44*CF; R²=0.43; Fisher's F=11.33, p<0.01). Symbols: □—successfully treated; Δ—unsuccessfully treated (treatment failure).

FIG. 5 shows the relationship of cadaverine fraction to the change in clinical attachment level mediated by therapy (deep scaling and root planing followed by prophylaxis in periodontitis patients, followed by scaling and prophylaxis every 3 months). CF after therapy is regressed on the change in mean clinical attachment level (ΔCAL). Symbols: □—periodontitis given the above treatment successfully (ΔCAL decreased within a year), Δ—periodontitis given the above treatment unsuccessfully (treatment failure) (ΔCAL increased within a year) and then treated with the new intensive protocol (ampicillin/metronizole for 2 weeks, weekly prophylaxis for 3 months, then scaling and prophylaxis every 3 months for a year), ∘—healthy subjects after scaling and prophylaxis every 3 months for a year.

A) Successfully treated.
B) Unsuccessfully treated (treatment failure) or healthy.

### DETAILED DESCRIPTION OF THE INVENTION

The present invention is a new method for identifying chronic periodontitis patients in whom conventional (standard) therapy is likely to be successful or is likely to fail (be unsuccessful), i.e. periodontitis patients referred to as being refractory to this form of therapy. The invention uses a measurement of the cadaverine mole fraction (CF) in a whole mouth plaque sample. Additionally, it requires measurements that are commonly made before therapy is begun and to follow the therapeutic response: age, fraction of gingival margins that bleed on probing (BOP) and the clinical attachment level (CAL). These measurements are incorporated into a discriminant algorithm that specifies whether a subject with mild to moderate periodontitis will be successfully treated or unsuccessfully treated by conventional therapy. In addition, if the subject is predicted to be unsuccessfully treated, a CF value of less than 0.45 indicates that a new intensive therapy developed for such patients (Haffajee et al. 2004) should be successful.

At present, unsuccessfully treated patients often endure many years of repeatedly failing therapy (Socransky et al. 2002). The present method is a completely new diagnostic procedure which is user friendly to periodontists, who are trained in the importance of obtaining accurate attachment level and bleeding on probing measurements. For them, collecting a sample of bacterial biofilm and mailing it for estimation of cadaverine and lysine would be simple. Indeed, cadaverine and lysine are stable. Samples that were mailed overnight at room temperature gave cadaverine fractions virtually identical to a second set of samples from the same patients mailed frozen on dry ice. The present invention therefore enables periodontists or other professionals in the field to predict when a patient is likely to fail (or succeed in) conventional (standard) therapy, based on measures of age, bleeding on probing, clinical attachment level, and the plaque cadaverine fraction in a whole mouth plaque sample, all obtained at the initial visit or a few months after beginning conventional therapy.

### EXAMPLES

### Description of the Periodontitis and Control Groups

The subjects were selected from participants in a study of response to therapy for chronic periodontitis at a periodontology clinic (Forsyth Institute, Boston) and were attending for routine maintenance. All were 21 or more years of age and had 20 or more teeth present. They were not pregnant and did not have any systemic or local condition that might have affected chronic periodontitis. They had originally presented for therapy by having more than 8 sites with pocket depths greater than 4 mm and a clinical attachment level greater than 3 mm. There were two sets of controls: a) healthy subjects with minimal gingivitis and minimal periodontitis (fraction of sites bleeding on gentle probing less than 12%; no sites with pocket depth or attachment level greater than 3 mm); and b) subjects not receiving any therapy or oral hygiene maintenance and attending the Oral Surgery Clinic, University of Oklahoma Health Sciences Center.

Each subject consented to participate in the project by signing a form approved by the local Institutional Review Board. The form explained the nature of the participation by asking consent to obtain a plaque sample and, in the case of the Forsyth subjects, permission to release previously recorded clinical measurements for this study. The request to Forsyth subjects was made a few days before a routine scheduled appointment and to Oklahoma subjects awaiting examination.

Table 1 shows that the percentage of women was similar in the groups responding successfully or unsuccessfully (treatment failure) to a standard, conventional therapy for chronic periodontitis (paragraph 0044 below; Chi-square statistic=1.45). By contrast, the healthy group was all female and significantly younger (ANOVA F statistic=8.67, p<0.01). Subjects who responded poorly to the conventional therapy described below (in paragraph 0044) had significantly more missing teeth than healthy subjects (ANOVA F statistic=3.76, p<0.05). Subjects responding well to this therapy did not differ from either healthy or poorly responsive subjects. No subject lost a tooth while enrolled in a protocol in this study. Many periodontitis subjects who failed to respond successfully to the conventional therapy were current cigarette smokers (63%) similar to 57% reported previously (Haffajee et al. 2004). No unsuccessfully treated subject, but almost half of successfully treated subjects had stopped smoking 9 to 25 years before entering the protocol. None of the healthy subjects were past or current smokers.

TABLE 1
Characteristic of the subject groups

| | Treatment | | | | | |
|---|---|---|---|---|---|---|
| | Failure¹ | | Successful¹ | | Healthy | |
| Measure | mean (S.D.)² | | mean (S.D.) | | mean (S.D.) | |
| Number of subjects | 8 | | 9 | | 5 | |
| Females | 5 | | 3 | | ³5 | |
| Age (Years) | 54.13 | (11.05) | 56.67 | (5.59) | 37.80 | (7.85) |
| Missing teeth | 5.63 | (4.75) | 3.11 | (2.67) | 0.40 | (0.55) |
| Current smokers (%) | 75.0 | | 0.0 | | 0.0 | |
| Past smokers | 0.0 | | 44.4 | | 0.0 | |
| Cadaverine fraction | 0.45 | (0.21) | 0.50 | (0.15) | 0.44 | (0.12) |

¹Periodontitis subjects' therapeutic responses were failure or success with the ‘standard’ protocol. Measurements for failed patients were obtained at enrollment in the intensive protocol (see Methods).
²Standard deviation
³Male healthy subjects not available; additional female provided pre-therapy measurements without participating in the protocol.

Plaque Sampling and Measurement of its Lysine and Cadaverine Contents

Volunteers from the Forsyth clinic agreed not to clean their teeth on the morning before a maintenance visit. Plaque was collected from the gingival crevice area of all the teeth using a curette. Samples with gross blood contamination were discarded. The plaque was transported to Oklahoma on dry ice and stored at −80° C.

The subjects recruited in Oklahoma were not in a plaque control program and the greatest amount of sample was obtained from sites near decayed or impacted teeth requiring therapy, or on the opposing teeth. Most exhibited mild to moderate gingivitis and a few also some loss of periodontal attachment, but detailed clinical periodontal examination of the Oklahoma subjects was not practicable.

Because the samples from different teeth surfaces were not mixed, some subjects provided two pools of plaque from different teeth surfaces. All or part of each subject's plaque was diluted with a 10-fold excess by weight of ice-cold 0.07 M NaCl in a 1.5 ml microfuge tube. After extraction using a Kontes Pellet Pestle, the supernatant fluid was centrifuged, cleared of protein by adding ice-cold trichloroacetic acid (TCA) to 10% w/v, re-centrifuged and diluted to 0.2 ml with distilled water. The TCA was removed by extracting it three times with 0.6 ml of ethyl acetate. The aqueous layer was air-dried, redissolved in 0.08 ml of 0.01 M HCl and applied to a 3×250 mm column of Spherogel (AA-NA+) using a Beckman System Gold Model 126 HPLC/Amino Acid Analyzer.

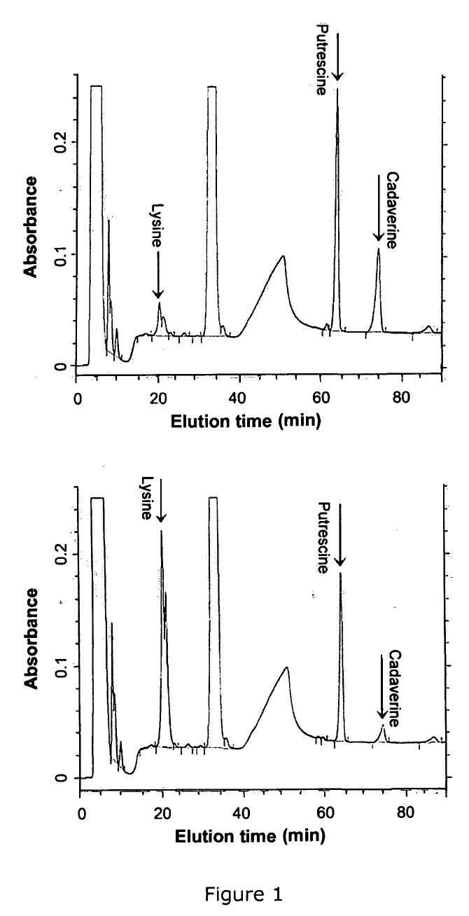

Lysine and cadaverine eluted in the second of a two buffer system using the column at 66° C. (first: 0.2 M Na citrate pH 5.55 in 0.3 M NaCl; second: pH 5.63 in 2.3 M NaCl) as described previously (Villanueva & Adlakha 1978). FIG. 1 shows a typical elution profile. After conversion of the areas of the appropriate, respective peaks to nmol cadaverine and nmol lysine (see legend to FIG. 1), the total amounts in each sample were calculated from the ratio of initial to recovered volume of sample applied to the column and normalized to mg wet weight of plaque assayed. Cadaverine fraction was determined as [nmol cadaverine]/([nmol cadaverine]+[nmol lysine]).

Cadaverine Fraction Reliability

The plaque cadaverine fraction from each subject ranged from 0.26-0.64 in five healthy subjects and 0.25-0.57 in five successfully treated periodontitis subjects. The average variation about each mean (duplicate estimations) was 11% (range, 0 to 26%). In samples from 3 unsuccessfully treated subjects taken a year apart, the plaque cadaverine fraction ranged from 0.06-0.52 with an average variation of 13% (range, 12 to 17%) and no change with time. The least amount of plaque assayed was 2.4 mg from an unsuccessfully treated patient who provided another 22.3 mg for assay a year later and whose respective cadaverine fractions were 0.055 and 0.065, a variation similar to that of the other subjects. Thus, the cadaverine fraction of plaque from periodontitis and healthy subjects was reasonably constant.

Compared with Forsyth subjects, Oklahoma subjects had a similar mean mass of plaque assayed and a similar lysine plus cadaverine content (nmol) per mg plaque, but a significantly lower cadaverine fraction (Table 2). Because differences in mean plaque cadaverine fraction within the Forsyth groups were not significant (Table 1, last line; ANOVA F statistic=0.27), the poor oral hygiene in the Oklahoma subjects may explain their lower cadaverine fraction (as discussed further below).

TABLE 2
Measurement of cadaverine and lysine in plaque
from Oklahoma and Forsyth subjects

| Group | Oklahoma | | | Forsyth | | |
|---|---|---|---|---|---|---|
| Measurement | Mean | s.d. | No. | Mean | s.d. | No. |
| Mean wet weight | 13.40 | 10.16 | 33 | 16.94 | 13.37 | 23 |
| of plaque sampled^{1,2} | | | | | | |
| (mg) | | | | | | |
| Lysine plus | 1.80 | 1.24 | 33 | 1.63 | 0.91 | 23 |
| cadaverine² (ng/mg) | | | | | | |
| Mean cadaverine | 0.32 | 0.19 | 33 | 0.46 | 0.16 | 23 |
| fraction² | | | | | | |

¹Sampled amount not necessarily the total amount collected per subject.
²Difference between Forsyth and Oklahoma groups for amount of plaque used in the assay, ‘t’ statistic = 1.13, p not significant; for lysine plus cadaverine/mg plaque, ‘t’ statistic = 0.57, p not significant; for cadaverine fraction [ng cad/(ng cad + ng lys)], ‘t’ statistic = 2.95, p < 0.01.

Clinical Measurements

Calibrated examiners at the Forsyth clinic recorded measurements at 6 surfaces per tooth: mesiobuccal, buccal, distobuccal, mesiolingual, lingual and distolingual to a maximum of 168 sites (third molars excluded). Measurements were visible plaque score, PS (0 or 1), bleeding on probing, BOP (0 or 1), probing pocket depth (PD) in mm, and clinical attachment level (CAL) in mm. PD and CAL were measured using a North Carolina probe with mm intervals (Hu-Friedy®, Chicago, Ill., USA). The percentage of sites with PS=1 and BOP=1 were tabulated for each subject. CAL was the distance from the enamel-cemental junction to the epithelial attachment in mm; PD plus recession to the lip of the pocket, or PD minus the distance from the free gingival margin to the enamel-cemental junction. Duplicate measurements were averaged across all sites on all teeth to give a single PD and CAL value Measurements were made pre-therapy and at the beginning of maintenance visits every 3 months (per Colombo et al. 1998, and Haffajee et al. 2004).

Treatment Protocols

All periodontitis patients were first treated by a standard, conventional protocol (Colombo et al. 1998). They received deep scaling and root-planing under local anesthetic (one quadrant a week for 4 weeks), after which prophylaxis and detailed home care instructions were given every 3 months for one year. Mean CAL was calculated pre-therapy and just before each 3-month maintenance visit. If mean CAL decreased relative to the pre-therapy CAL at all four visits, treatment of the subject was considered to be successful. If mean CAL was increased at any one of these maintenance visits, the subject was classified as unsuccessfully treated (treatment failure).

All of the successfully treated patients and three unsuccessfully treated (treatment failure) subjects were identified by this protocol at the Forsyth. Five additional unsuccessfully treated patients were identified similarly by outside, local practitioners. Following a new baseline clinical examination, all 8 unsuccessfully treated subjects participated in a new, intensive protocol of aggressive combined antibiotic/oral hygiene therapy (per Haffajee et al. 2004). In this protocol, after baseline monitoring, patients received SRP, locally delivered tetracycline at pockets >4 mm, systemically administered amoxicillin (500 mg, t.i.d. for 14 days)+metronidazole (250 mg, t.i.d. for 14 days), and professional removal of supragingival plaque weekly for 3 months. Clinical measurements were made at the last visit for weekly cleaning and every 3 months thereafter for two years, but the measurements were reported to have essentially stabilized after one year (Haffajee et al. 2004).

All but one of 6 healthy volunteers received scaling as necessary, prophylaxis, and re-enforcement of home care procedures every 3 months. Clinical measurements were taken at each visit until one year after enrollment (year-long protocol).

Data Analysis

Statistical analysis was performed using an XLSTAT computer program (Addinsoft, New York, N.Y.). The unit of analysis was the subject, whose measurements of extent of plaque score, bleeding on probing, pocket depth, and clinical attachment level were classified by therapy (pre- and post therapy values) and group (successfully treated, unsuccessfully treated, or healthy) in a 2-way ANOVA. If a significant difference was detected in the ANOVA, Duncan's test was used to identify which classifications differed significantly. Within each group and also in combined groups as appropriate, each pre-therapy clinical attachment level or its difference from post-therapy, the outcome variable (dependent), was regressed on plaque cadaverine fraction alone, or with other variables including age. Squared regression coefficients were adjusted for small numbers and multiple correlations (R²) to provide estimates of relationship strength.

Table 3 shows the effects of therapy and group on the clinical findings. Mean plaque scores did not differ by group or following therapy (Table 3a; PII-ANOVA F statistic=0.351, d.f.=5.38, not significant). Pre-therapy BOP was significantly greater in periodontitis subjects than healthy subjects, and only decreased significantly post-therapy in unsuccessfully treated (treatment failure) subjects (Table 3b; BOP-ANOVA F statistic=9.94, d.f.=5.38, p<0.001; Duncan's test p<0.02). Similarly, pre-therapy PD was significantly greater in periodontitis subjects than healthy subjects, and decreased significantly in unsuccessfully treated (treatment failure) subjects (Table 3c; PD-ANOVA F statistic=6.28, d.f.=5.38, p<0.001; Duncan's test p<0.04). Pre-therapy CAL was significantly less in healthy subjects than in periodontitis subjects, but the reductions following therapy were not significant in any group (Table 3d; CAL-ANOVA F=7.57, d.f.=5.38, p<0.001; Duncan's test p<0.01). In successfully treated subjects, the small decrease in CAL following therapy may have been obscured by the large subject variation (Table 3d). In unsuccessfully treated (treatment failure) subjects following the aggressive combined antibiotic/oral hygiene therapy discussed above, the decrease in CAL in some subjects was nullified by the increase in others.

TABLE 3
Effect of group and therapy on PS, BOP, PD and
CAL. Different letters indicate mean differences
with at least a 95% confidence level.
A: Mean plaque scores (PS) pre- and post-therapy by group.

| Therapy | Group | Mean PS (S.D.) | Duncan's test |
|---|---|---|---|
| Pre | Healthy | 34.60 (22.49) | A |
| Post | Healthy | 49.00 (15.25) | A |
| Pre | Successful | 45.44 (24.11) | A |
| Post | Successful | 40.67 (21.45) | A |
| Pre | Failure | 49.25 (28.73) | A |
| Post | Failure | 46.50 (21.94) | A |

B: Mean BOP scores pre- and post-therapy by group.

| Therapy | Group | Mean PS (S.D.) | Duncan's test | |
|---|---|---|---|---|
| Pre | Healthy | 4.60 (3.91) | | B |
| Post | Healthy | 5.00 (2.65) | | B |
| Pre | Successful | 31.00 (14.53) | A | |
| Post | Successful | 21.22 (8.09) | A | |
| Pre | Failure | 22.25 (14.02) | A | |
| Post | Failure | 4.13 (3.91) | | B |

C: Mean PD pre- and post-therapy by group.

| Therapy | Group | Mean PS (S.D.) | Duncan's test | | |
|---|---|---|---|---|---|
| Pre | Healthy | 2.15 (0.15) | | B | C |
| Post | Healthy | 1.90 (0.18) | | | C |
| Pre | Successful | 3.05 (0.61) | A | | |
| Post | Successful | 2.70 (0.59) | A | B | |
| Pre | Failure | 3.05 (0.55) | A | | |
| Post | Failure | 2.44 (0.25) | | B | C |

D: Mean CAL pre- and post-therapy by group.

| Therapy | Group | Mean PS (S.D.) | Duncan's test | |
|---|---|---|---|---|
| Pre | Healthy | 1.54 (0.57) | | B |
| Post | Healthy | 1.39 (0.30) | | B |
| Pre | Successful | 3.60 (0.88) | A | |
| Post | Successful | 3.31 (0.82) | A | |
| Pre | Failure | 3.86 (1.55) | A | |
| Post | Failure | 3.55 (0.91) | A | |

Discrimination Between Healthy, Successful, and Unsuccessful Responses to Standard, Conventional Therapy

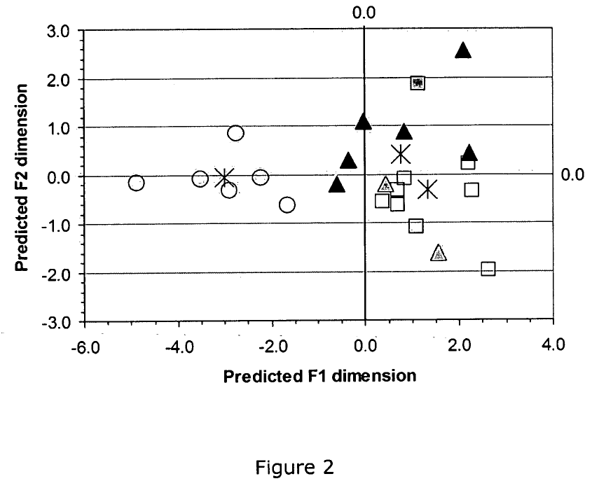

In the three groups (healthy, successful, and unsuccessful, i.e., failure) pretherapy measurements of Age, BOP and CAL and a posttherapy measurement of CF gave rise to two discriminant functions, which differentiated each individual by how these independent variables related to each group. A discriminant function formula has the form, L=b₁x₁+b₂x₂+ . . . +bₙxₙ+c, where x₁ is Age, x₂ is BOP, x₃ is CAL, x₄ is CF. The coefficients (b₁, b₂ . . . ) are discriminant coefficients, similar to regression coefficients that reflect the unique contribution to group classification of each independent variable (Age etc.) adjusted for each of the other variables. Together with the regression constant (c), it gives L, the value from applying the formula to data from a given individual. A canonical plot is created, in which the two axes are the two discriminant functions. An X within the plot locates the centroids for each group (FIG. 2). These centroids are the mean discriminant scores for each of the group categories for each of the two discriminant functions. FIG. 2 shows that healthy groups are located in a different space from the two periodontitis (successful and treatment failure) groups. The group centroid that is least distant from a discriminant score point using the generalized distance function derived from Mahalanobis distance (D²) identifies an individual's classification. Any other appropriate statistical analysis package able to perform discriminant analysis as contemplated herein can be used herein, such as, but not limited to, SPSS™ SAS™, Minitab™, S-Plus™ and Statsoft™.

Eigenvalues are a set of statistics that quantify the variation in a group of variables and its accountability by one of the categories (e.g., healthy). There is one eigenvalue for each discriminant function. The ratio of the eigenvalues indicates the relative discriminating power of the discriminant functions. In this study, the first discriminant function (F1) accounted for 97% and the second (F2) for 3% of the power of the model. Wilks' lambda is a measure of the significance of the difference the centroid of the means for each of the three groups on the independent variables (Age etc.). Lambda varies from 0 to 1; the closer to 0, the better is the differentiation of the groups. The associated F-statistic is then computed according to the Rao's approximation to obtain the significance of lambda.

The equation algorithms are:

F1=−9.336976633+0.075257305*Age+0.033090462*BOP+0.789805982*CAL+5.047008391*CF.

F2=−0.539517856−0.002877868*Age−0.059588699*BOP+0.678545658*CAL−0.429365657*CF

The centroid points are:
Healthy: F1=−2.999, F2=−0.069
Unsuccessful (treatment failure): F1=0.770, F2=0.410
Successful: F1=1.315, F2=−0.318

These above centroids and canonical discriminant function coefficients determine F1 and F2 scores for each individual, and therefore a point on the canonical structure matrix whose distance can be compared with the respective centroids to classify a patient from age, CF and pre-therapy clinical measurements (BOP and CAL). FIG. 2 shows the results from the 23 subjects and the three centroids. The variables correctly assigned all the healthy patients, all but one of the nine successfully treated patients and all but two of the eight unsuccessfully treated (treatment failure) patients, an accuracy of 82% (Wilks' Lambda=0.19, F=5.49, d.f.1=8, d.f. 2=34, one-tailed p<0.001). If CF was omitted, 4 of the 9 successfully treated subjects were misclassified as unsuccessful (treatment failure). Discrimination between successful and unsuccessful (treatment failure) groups was less accurate if mean PD and/or plaque fraction were included and not significant if the healthy group or additional variables were omitted. In the present invention, any alternative set of discriminant function equations and corresponding centroid points can be used which have been computed from an appropriate database of patients who have been treated with the standard treatment regimen as described herein.

Relevant Studies

Cadaverine Fraction and Pre-Therapy Measurements

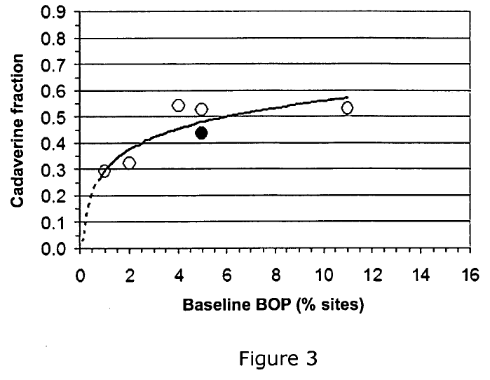

In healthy subjects, the plaque cadaverine fraction was significantly associated with the logarithm of percent sites that bled on probing pre-therapy, but not with post-therapy bleeding on probing, or the percent difference in bleeding on probing from pre-therapy. The significant relationship with pre-therapy bleeding on probing was also apparent in a healthy subject who did not participate subsequently in the ‘year-long’ protocol (FIG. 3). At a maximal plaque cadaverine fraction of 0.55, 12% of sulcular sites bled on probing, falling to zero if plaque cadaverine fraction was extrapolated to zero (FIG. 3). The derived regression equation and the associated statistics are CF=0.26*Log(BOP)+0.29; adjR2=0.67, p<0.03.

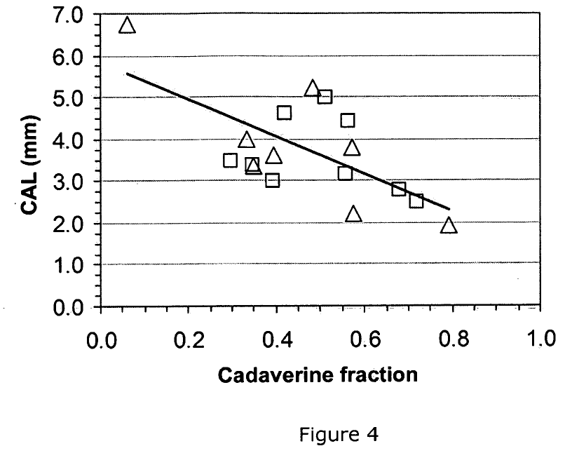

Within the periodontitis subjects, mean clinical attachment level pre-therapy was negatively related to plaque cadaverine fraction (FIG. 4). Multiple regression indicated that healthy subjects were also associated with plaque cadaverine fraction if adjustments were made for pre-therapy pocket depth, age, and bleeding on probing, but not for visible plaque score. The association with pocket depth accounted for 75% of the variance and the others each accounted for 5%. (Equation: CAL=−3.73+2.19*PD−1.69*CF+0.04*Age−0.04*BOP; total adjR²=0.90; d.f=4.18; F=51.02, p<0.001; partial adjR² for PD=0.75, p<0.0001; partial adjR² for CF, Age and BOP each 0.05, p<0.02). If the association with plaque cadaverine fraction was omitted, relationships were unchanged, except that total adjR² was reduced to 0.85. If either age or PD were also omitted, the association of CAL with BOP became insignificant. Not considering both age and PD, resulted in CAL becoming positively and more strongly associated with BOP (Equation: CAL=3.84+0.04*BOP−3.39*CF; total adjR²=0.32; d.f=2.20; F=6.13, p<0.01; partial adjR² for BOP=0.20, p<0.02; partial adjR² for CF=0.12, p<0.05). Nevertheless, the multiple association using BOP and CF was much weaker than using age and PD as independent variables.

Relationship of Plaque Cadaverine Fraction to Post-Therapy Changes in Attachment Level

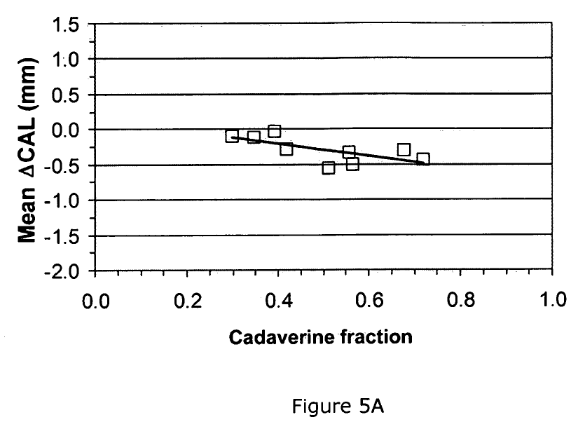

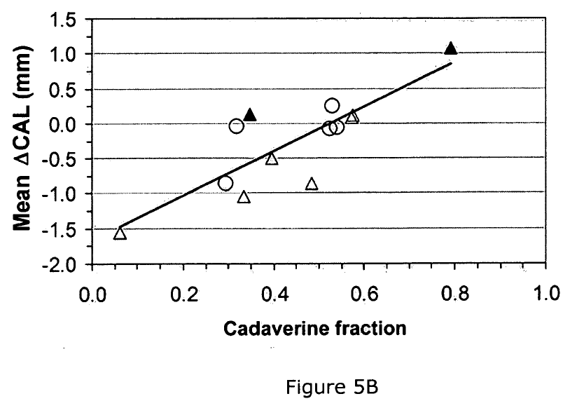

In successfully treated subjects, the decrease in ΔCAL was dependent on plaque cadaverine fraction (FIG. 5a; ΔCAL=0.15−0.88*CF; adjR²=0.40, p<0.04). Adjusting for pre-therapy CAL and age (multiple regression), respectively added to and decreased the effects of plaque cadaverine fraction. ΔCAL became more negative as CAL increased and less negative as age increased (ΔCAL=0.05−1.00*CF−0.11*CAL+0.01*Age; total adjR²=0.95; d.f.=3.5; F=58.2, p<0.001. Partial adjR²−age=0.42, p<0.03; partial adjR²−CF=0.31, p<0.001; partial adjR²−CAL=0.22, p<0.01).

In unsuccessfully treated (treatment failure) subjects, ΔCAL was strongly and positively related to plaque cadaverine fraction after the intensive protocol, (FIG. 5b; ΔCAL=−1.80+3.34*CF; adjR²=0.71, p<0.01). Surprisingly, healthy subjects displayed a similar relationship although not significant (ΔCAL=−1.17+2.31*CF; adjR²=0.31). Combining the two groups retained this relationship (adjR²=0.67, p<0.01), and multiple regression indicated a significant additional association with pre-therapy PD that decreased ΔCAL (ΔCAL=−0.29+2.56*CF−0.41*PD; total adjR²=0.77; d.f.=10.2; F=20.5, p<0.001). However, most of the variance was due to the cadaverine fraction association (partial adjR²−CF=0.64, p<0.01; partial adjR²−PD=0.13, p<0.04).

Unsuccessfully treated (treatment failure) and healthy subjects were divided by whether CAL had decreased or increased post-therapy. The mean plaque cadaverine fraction of the attachment losers (4 unsuccessfully treated in the standard protocol and 1 healthy) was 0.563 (0.158 standard deviation) and that of the attachment gainers (4 unsuccessfully treated in the standard protocol and 4 healthy) was 0.369 (0.157 standard deviation). This difference was of borderline significance in the unpaired ‘t’ test (‘t’=2.17; p=0.05), but significant in the Mann-Whitney test (U=46.667; p=0.03). These findings suggest that any kind of oral hygiene therapy does not support re-attachment in healthy and unsuccessfully treated (treatment failure) subjects whose pre-therapy plaque cadaverine fraction is greater than about 0.45; i.e. when about half or more of the lysine is depleted.

Significance and Value

The presence of cadaverine in plaque and its source in lysine are well known (Hyatt & Hayes 1975). The fraction of lysine (substrate) plus cadaverine (product) that was product (plaque cadaverine mole fraction, CF) was therefore taken as a measure of lysine decarboxylase activity. Lysine decarboxylase is made by E. corrodens and some Capnocytophaga spp. (Holmes et al. 1995) which co-colonize from saliva in healthy individuals. These bacteria are present as 1-5% of the plaque microbiota in healthy adults within 2-6 h of abstaining from oral hygiene (Li et al. 2004), and at about 3% in both health and disease thereafter (Ximenez-Fyvie et al. 2000).

Forsyth subjects were consciously maintaining effective oral hygiene, whereas the Oklahoma subjects were clearly not doing so and likely had more crevicular fluid exudate (inflammation) and more anaerobic, gram negative bacteria and spirochetes in their plaque (mature plaque). The result is more lysine from crevicular fluid and more bacterial protein metabolism in gingival pockets or sulci. Moreover, the bacteria associated with lysine decarboxylase production do not appear to be increased in disease-associated plaque (Ximenez-Fyvie et al. 2000), and a smaller cadaverine fraction was present in the plaque from Oklahoma subjects. Correspondingly, the smallest plaque cadaverine fraction was from subjects who had experienced the greatest periodontal attachment level (greatest CAL) prior to therapy.

Given the small number of subjects in each Forsyth group, it would be expected that regressions involving plaque cadaverine fraction would be insignificant unless the proposed relationships are present. Most healthy adults exhibit a few sites that bleed on probing (Goodson et al. 2004); perfect gingival health is generally agreed to be rare. The plaque cadaverine fraction is proposed to measure lysine depletion and therefore the extent of crevicular fluid induction. Subjects in whom the plaque cadaverine fraction is greater would require more crevicular fluid to replenish lysine and more sites would be predisposed to plaque maturation and bleeding on probing. Thus, a greater plaque cadaverine fraction and crevicular fluid flow decreases cleaning efficacy and associates with more sites exhibiting bleeding on probing at the initial examination.

After a year of promoting intensive oral hygiene in healthy subjects, the number of sulci that bled on probing remained the same but there was no association with plaque cadaverine fraction. Because a subject who did not participate in the protocol contributed also to the pre-therapy relationship, it is unlikely that the plaque cadaverine fraction was altered by the oral hygiene protocol. More likely, the additional and possibly unnecessary attention to oral hygiene prevented plaque maturation at the expense of mild trauma, creating sites that bled on probing independently of plaque cadaverine fraction.

The successfully treated periodontitis subjects had about 18 months of oral hygiene before the plaque cadaverine fraction was obtained. Within these subjects, a greater plaque cadaverine fraction would cause greater lysine starvation of dentally attached cells and therefore induce a greater crevicular fluid exudate. Although the increase in crevicular fluid replenishes lysine and promotes re-attachment, gram negative bacteria and spirochetes may have remained in the oral cavity in greater numbers than in healthy subjects. Despite the oral hygiene control, the plaque tends to mature faster than in healthy subjects (Socransky & Haffajee 2005) and more sites bled on probing than in healthy subjects.

In unsuccessfully treated (treatment failure) and healthy subjects, however, the results indicate that a greater plaque cadaverine fraction hinders reattachment after therapy. The positive association of plaque cadaverine fraction with attachment level increase these subjects compared with the negative relationship in successfully treated periodontitis subjects is important. It suggests that the host response to lysine depletion in unsuccessfully treated (treatment failure) subjects resembles that of healthy subjects, a completely unexpected finding. The similarity to healthy subjects also appears true for bleeding on probing. The mean fraction of sites that bled on probing after therapy in unsuccessfully treated (treatment failure) subjects resembled that of healthy subjects and was significantly less than in successfully treated subjects.

A high plaque cadaverine fraction does not appear likely to predispose healthy subjects to attachment loss, because the subjects exhibiting more disease (increased clinical attachment level) displayed a smaller plaque cadaverine fraction. Correspondingly, not maintaining oral hygiene lowers the cadaverine fraction of plaque, as discussed above.

In this study, an increased cadaverine fraction (greater lysine depletion) resulted in a greater attachment gain in the successfully treated subjects. By contrast, in the unsuccessfully treated (treatment failure) periodontitis and healthy subjects, clinical attachment level tended to increase if the plaque cadaverine fraction (CF) was >45%. This result suggests inadequate replenishment of lysine-depleted cells by the exudation of crevicular fluid from the sulcus due to little proinflammatory interleukin stimulation in these subjects and consistent with the low BOP observed after therapy. The lack of crevicular fluid association with a high plaque cadaverine fraction would deplete the DAT cells of lysine and causes the clinical attachment level to increase despite therapy. This lack of crevicular fluid may also cause that abnormal secular microbiota that characterizes unsuccessfully treated (treatment failure) periodontitis subjects (Socransky et al. 2002).

Utility

In one embodiment of the invention, a prediction of a treatment outcome of a periodontitis patient to a standard treatment regimen is based on the subject's age, BOP and CAL measurements prior to therapy plus the plaque CF (cadaverine mole fraction of the lysine plus cadaverine contents derived from a whole mouth sample of plaque). The plaque CF may be obtained at initial examination or 3 to 6 months after the patient has completed the deep scaling portion of conventional therapy and been placed on maintenance therapy. Measurements may then be restricted only to those patients who are suspected of not responding successfully to the therapy. Inserting these measurements into equation algorithms to determine F1 and F2 as described above for example and then determining whether the point (F1, F2) is closer to the centroid points for unsuccessfully treated (treatment failure) or successfully treated subjects predicts when a subject having periodontitis is likely to be successfully treated by standard, conventional therapy or when a subject having periodontitis is likely to have an unsuccessful (i.e., treatment failure) outcome to standard therapy.

In a second embodiment, if a patient has already been found to have been unsuccessfully treated with the standard conventional therapy (i.e., treatment failure), a CF value of greater than 0.45 will predict that even subsequent aggressive combined antibiotic/oral hygiene therapy is unlikely to be successful.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the methods and steps described in the specification. As one of ordinary skill in the art will readily appreciate from the disclosure of the present invention, methods, or steps, presently existing or later to be developed that perform substantially the same function or achieve substantially the same result as the corresponding embodiments described herein may be utilized according to the present invention. Accordingly, the appended claims are intended to include within their scope such processes, methods, or steps.

All articles, publications, patents, or published applications cited herein are expressly incorporated herein by reference in their entireties. Further, each of U.S. Pat. Nos. 6,103,220; 6,187,296; 6,576,435; and 6,974,700 is expressly incorporated herein by reference in its entirety.

### CITED REFERENCES

Albandar, J. M., Brunelle, J. A. & Kingman, A. (1999) Destructive periodontal disease in adults 30 years of age and older in the United States, 1988-1994. J. Periodontol. 70, 13-29.
Armitage, G. C. (1996) Periodontal diseases: diagnosis. Ann. Periodontol. 1, 37-215.
Bickel, M., Munoz, J. L. & Giovannini, P. (1985) Acid-base properties of human gingival crevicular fluid. J Dent. Res. 64, 1218-1220.
Boyer, B. P., Ryerson, C. C., Reynolds, H. S., Zambon, J. J., Genco, R. J. & Snyder, B. (1996) Colonization by Actinobacillus actinomycetemcomitans, Porphyromonas gingivalis and Prevotella intermedia in adult periodontitis patients as detected by the antibody-based Evalusite Test. J Clin. Periodontol 23, 477-484.
Colombo, A. P., Haffajee, A. D., Dewhirst, F. E., Paster, B. J., Smith, C. M., Cugini, M. A. & Socransky, S. S. (1998) Clinical and microbiological features of refractory periodontitis subjects. J. Clin. Periodontol. 25, 169-180.
Colombo, A. P., Haffajee, A. D., Smith, C. M., Cugini, M. A. & Socransky, S. S. (1999) Discrimination of refractory periodontitis subjects using clinical and laboratory parameters alone and in combination. J. Clin. Periodontol. 26, 569-576.
Dinarello, C. A. (1996) Biologic basis for interleukin-1 in disease. Blood 87, 2095-2147.
Furuichi, Y., Lindhe, J., Ramberg, P. & Volpe, A. R. (1992) Patterns of de novo plaque formation in the human dentition. J Clin. Periodontol 19, 423-433.
Goldman, M. J., Ross, I. F. & Goteiner, D. (1986) Effect of periodontal therapy on patients maintained for 15 years or longer. A retrospective study. J Periodontol 57, 347-353.
Goodson, J. M., Palys, M. D., Carpino, E., Regan, E. O., Sweeney, M. & Socransky, S. S. (2004) Microbiological changes associated with dental prophylaxis. J. Am. Dent. Assoc. 135, 1559-1564.
Griffiths, G. S., Wilton, J. M. & Curtis, M. A. (1997) Permeability of the gingival tissues to IgM during an experimental gingivitis study in man. Arch. Oral Biol. 42, 129-136.
Groves, R. W., Mizutani, H., Kieffer, J. D. & Kupper, T. S. (1995) Inflammatory skin disease in transgenic mice that express high levels of interleukin 1 alpha in basal epidermis. Proc. Natl. Acad. Sci. U.S.A 92, 11874-11878.
Haffajee, A. D., Cugini, M. A., Dibart, S., Smith, C., Kent, R. L., Jr. & Socransky, S. S. (1997) Clinical and microbiological features of subjects with adult periodontitis who responded poorly to scaling and root planing. J. Clin. Periodontol. 24, 767-776.
Haffajee, A. D., Uzel, N. G., Arguello, E. I., Torresyap, G., Guerrero, D. M. & Socransky, S. S. (2004) Clinical and microbiological changes associated with the use of combined antimicrobial therapies to treat “refractory” periodontitis. J. Clin. Periodontol. 31, 869-877.
Hirschfeld, L. & Wasserman, B. (1978) A long-term survey of tooth loss in 600 treated periodontal patients. J Periodontol 49, 225-237.
Holmes, B., Pickett, M. & Hollis, D. (1995) Unusual Gram-negative bacteria, including Capnocytophaga, Eikenella, Pasteurella and Streptobacillus. In Manual of clinical microbiology, ed. Murray P R, pp. 449-508. Washington D.C.: ASM Publications.
Holmlund, A., Hanstrom, L. & Lerner, U. H. (2004) Bone resorbing activity and cytokine levels in gingival crevicular fluid before and after treatment of periodontal disease. J Clin. Periodontol 31, 475-482.
Hyatt, A. T. & Hayes, M. L. (1975) Free amino acids and amines in human dental plaque. Arch. Oral Biol 20, 203-209.
Levine, M. (1985) The role for butyrate and propionate in mediating HeLa-cells growth inhibition by human dental plaque fluid from adult periodontal disease. Arch. Oral Biol 30, 155-159.
Levine, M., LaPolla, S., Owen, W. L. & Socransky, S. S. (2002) Antibody-based diagnostic for refractory periodontitis. J. Clin. Periodontol. 29, 935-943.
Levine, M. & Miller, F. C. (1991) Use of monoclonal antibodies with neutralizing effects on toxic antigens from human bacterial plaque to detect specific bacteria by colony blotting. J Clin. Microbiol. 29, 2809-2816.
Levine, M. & Miller, F. C. (1996) An Eikenella corrodens toxin detected by plaque toxin-neutralizing monoclonal antibodies. Infect. Immun. 64, 1672-1678.
Levine, M., Progulske-Fox, A., Denslow, N. D., Farmerie, W. G., Smith, D. M., Swearingen, W. T., Miller, F. C., Liang, Z., Roe, B. A. & Pan, H. Q. (2001) Identification of lysine decarboxylase as a mammalian cell growth inhibitor in Eikenella corrodens: possible role in periodontal disease. Microb. Pathog. 30, 179-192.
Li, J., Helmerhorst, E. J., Leone, C. W., Troxler, R. F., Yaskell, T., Haffajee, A. D., Socransky, S. S. & Oppenheim, F. G. (2004) Identification of early microbial colonizers in human dental biofilm. J. Appl. Microbiol. 97, 1311-1318.
Loe, H., Theilade, E. & Jensen, S. B. (1965) Experimental gingivitis in man. pp. 177-187.
Loe, H. & Holm-Pedersen, P. (1965) Absence and presence of fluid from normal and inflamed gingivae. Periodontics. 3, 171-177.
Loesche, W. J., Giordano, J. & Hujoel, P. P. (1990) The utility of the BANA test for monitoring anaerobic infections due to spirochetes (Treponema denticola) in periodontal disease. J Dent. Res. 69, 1696-1702.
Loos, B. G., Craandijk, J., Hoek, F. J., Wertheim-van Dillen, P. M. & van, d., V (2000) Elevation of systemic markers related to cardiovascular diseases in the peripheral blood of periodontitis patients. J Periodontol 71, 1528-1534.

Marsh, P. D. (2003) Are dental diseases examples of ecological catastrophes? Microbiology 149, 279-294.
McAnally, J. R. & Levine, M. (1993) Bacteria reactive to plaque-toxin-neutralizing monoclonal antibodies are related to the severity of gingivitis at the sampled site. Oral Microbiol. Immunol. 8, 69-74.
McFall, W. T., Jr. (1982) Tooth loss in 100 treated patients with periodontal disease. A long-term study. J Periodontol 53, 539-549.
Niederman, R., Buyle-Bodin, Y., Lu, B. Y., Naleway, C., Robinson, P. & Kent, R. (1996) The relationship of gingival crevicular fluid short chain carboxylic acid concentration to gingival inflammation. J Clin. Periodontol 23, 743-749.
Nieminen, A., Siren, E., Wolf, J. & Asikainen, S. (1995) Prognostic criteria for the efficiency of non-surgical periodontal therapy in advanced periodontitis. J. Clin. Periodontol. 22, 153-161.
Offenbacher, S., Collins, J. G. & Arnold, R. R. (1993) New clinical diagnostic strategies based on pathogenesis of disease. J Periodontal Res. 28, 523-535.
Parameters of care supplement (2000) Parameter on chronic periodontitis with slight to moderate loss of periodontal support. J Periodontol 71, 853-855.
Persson, G. R., Alves, M. E., Chambers, D. A., Clark, W. B., Cohen, R., Crawford, J. M., DeRouen, T. A., Magnusson, I., Schindler, T. & Page, R. C. (1995) A multicenter clinical trial of PerioGard in distinguishing between diseased and healthy periodontal sites. (I). Study design, methodology and therapeutic outcome. J Clin. Periodontol 22, 794-803.
Salonen, J. I. (1994) Proliferative potential of the attached cells of human junctional epithelium. J. Periodontal Res. 29, 41-45.
Schroeder, H. E. & Listgarten, M. A. (1977) Fine structure of the developing epithelial attachment of human teeth. Tarrytown, N.Y.: S. Karger.
Socransky, S. S. & Haffajee, A. D. (2005) Periodontal microbial ecology. Periodontol. 2000. 38, 135-187.
Socransky, S. S., Smith, C. & Haffajee, A. D. (2002) Subgingival microbial profiles in refractory periodontal disease. J Clin. Periodontol 29, 260-268.
Stashenko, P., Fujiyoshi, P., Obernesser, M. S., Prostak, L., Haffajee, A. D. & Socransky, S. S. (1991) Levels of interleukin 1 beta in tissue from sites of active periodontal disease. J. Clin. Periodontol. 18, 548-554.
Tsalikis, L., Parapanisiou, E., Bata-Kyrkou, A., Polymenides, Z. & Konstantinidis, A. (2002) Crevicular fluid levels of interleukin-1alpha and interleukin-1beta during experimental gingivitis in young and old adults. J. Int. Acad. Periodontol. 4, 5-11.
Villanueva, V. R. & Adlakha, R. C. (1978) Automated analysis of common basic amino acids, mono-, di-, and polyamines, phenolicamines, and indoleamines in crude biological samples. Anal. Biochem. 91, 264-275.
Ximenez-Fyvie, L. A., Haffajee, A. D. & Socransky, S. S. (2000) Comparison of the microbiota of supra- and subgingival plaque in health and periodontitis. J Clin. Periodontol 27, 648-657.

What is claimed is:

1. A method of predicting an outcome of a treatment regimen for a patient having chronic periodontitis, comprising:
obtaining from the patient measurements of age, bleeding on probing, clinical attachment level, and plaque cadaverine fraction;
combining the measurements of age, bleeding on probing, clinical attachment level, and plaque cadaverine fraction using a mathematical algorithm comprising a set of discriminant function equations based on variables of age, bleeding on probing, clinical attachment level, and plaque cadaverine fraction thereby determining a score set for the set of discriminant function equations; and
using the score set to predict whether the outcome of the treatment regimen will be success or failure.

2. The method of claim 1 wherein the treatment regimen is a standard treatment regimen comprising deep scaling and root planing and home care.

3. The method of claim 2 wherein an outcome of success is based on a decreased mean clinical attachment level measurement at each of four clinical assessments taken at approximately three month intervals after the deep scaling and root planing of the standard treatment regimen.

4. The method of claim 2 wherein an outcome of failure is based on an increased mean clinical attachment level measurement at least one of four clinical assessments taken at approximately three month intervals after the deep scaling and root planing of the standard treatment regimen.

5. The method of claim 1 wherein the patient measurements are taken from the patient having chronic periodontitis prior to undergoing the treatment regimen.

6. The method of claim 1 wherein the set of discriminant function equations is based on a pretherapy data set obtained from a plurality of healthy individuals and chronic periodontitis patients who were subsequently treated successfully and chronic periodontis patients who subsequently failed treatment.

7. The method of claim 1 wherein in the step of using the score set to predict success or failure, the score set is compared to a centroid point corresponding to treatment success and to a centroid point corresponding to treatment failure, and wherein a prediction of treatment success is made when the score set is closest to the centroid point corresponding to treatment success, and wherein a prediction of treatment failure is made when the score set is closest to the centroid point corresponding to treatment failure.

8. The method of claim 1 wherein, when the outcome of the treatment regimen is predicted to be failure, and also when the plaque cadaverine fraction of the patient exceeds 0.45, the patient is further predicted to have an outcome of failure to an aggressive combined antibiotic/oral hygiene therapy.

9. The method of claim 1 wherein the plaque cadaverine fraction is [nmol/cadaverine/(nmol lysine+nmol cadaverine)] as measured from a plaque sample taken from the patient before or after deep scaling.

10. A method of predicting an outcome of an aggressive combined antibiotic/oral hygiene therapy regimen for a chronic periodontitis patient who already has a failed outcome to a conventional standard treatment regimen, comprising:
obtaining from the patient a measurement of plaque cadaverine fraction; and
predicting that the patient will have a failed outcome to an aggressive combined antibiotic/oral hygiene therapy regimen when the measurement of plaque cadaverine fraction exceeds 0.45.

11. The method of claim 10 wherein the plaque cadaverine fraction is [nmol/cadaverine/(nmol lysine+nmol cadaverine)] as measured from a plaque sample taken from the patient.
